(19) United States
(12) Patent Application Publication
(10) Pub. No.: US 2025/0072772 A1
(43) Pub. Date: Mar. 6, 2025

(54) METHOD FOR ESTIMATING AND CORRECTING HEART RATE IN EXERCISES USING BAROMETER SIGNAL FROM WEARABLE DEVICES

(71) Applicant: SAMSUNG ELETRÔNICA DA AMAZÔNIA LTDA., CAMPINAS (BR)

(72) Inventors: PAULA GABRIELLY RODRIGUES, CAMPINAS (BR); FRANK ALEXIS CANAHUIRE CABELLO, CAMPINAS (BR); ITALOS ESTILON DA SILVA DE SOUZA, CAMPINAS (BR); RUAN ROBERT BISPO DOS SANTOS, CAMPINAS (BR); OTAVIO AUGUSTO BIZETTO PENATTI, CAMPINAS (BR); DONGHYUN LEE, SUWON-SI (KR); JAEHWAN JUNG, SUWON-SI (KR)

(73) Assignee: SAMSUNG ELETRÔNICA DA AMAZÔNIA LTDA., CAMPINAS (BR)

(21) Appl. No.: 18/378,275

(22) Filed: Oct. 10, 2023

(30) Foreign Application Priority Data
Sep. 1, 2023 (BR) 102023017789 1

(51) Int. Cl. A61B 5/024; A61B 5/00

(52) U.S. Cl. CPC A61B 5/02416; A61B 5/6801; A61B 5/7207; A61B 5/7221; A61B 5/7239; A61B 5/7267; A61B 5/7275; A61B 2562/02

(57) ABSTRACT

A method that estimates and corrects the heart rate when the photoplethysmography signal is unreliable by using an exponential model with parameters adjusted with machine learning strategies using the reliable HR from the PPG signal and the value of the barometer signal collected by a wearable device after the end of a workout session.

### CROSS-REFERENCE TO RELATED APPLICATION(S)

This application is based on and claims priority under 35 U.S.C. § 119 to Brazilian Patent Application No. BR 102023017789-1, filed on Sep. 1, 2023, in the Brazilian Intellectual Property Office, the disclosure of which is incorporated by reference herein in its entirety.

### TECHNICAL FIELD

The present invention is related to the field of healthcare technology, health monitoring, and fitness applications for wearable devices and describes a method to estimate the heart rate (HR) of workouts when the photoplethysmography signal from wearable devices, such as smartwatches, is affected by motion artifacts or others type of noise. The method uses machine learning strategies to estimate the parameters of an exponential model after the end of a workout session using the reliable parts of the heart rate estimated from the PPG signal and replacing the remaining parts with an estimation based on the value of the barometer signal collected by a wearable device during an exercise.

The method allows a more accurate estimation of the heart rate in scenarios where there are differences in elevation, and consequently, improves all the other features based on heart rate information.

### BACKGROUND OF THE INVENTION

In recent years, the popularity of wearable devices has increased extensively. Especially in the case of smartwatches, this increase in demand is due to their ability to easily get information about peoples' health, such as data related to sleep quality, level of activity during the day, stress, and changes in heart rate during an exercise. Heart rate monitoring is one of the core features of this kind of device. The heart rate is estimated by a non-invasive and low-cost optical technique called photoplethysmography (PPG), which uses LEDs and photodiodes to measure oscillations in blood volume that can be translated into the frequency of the heartbeats.

Considering the nature of the PPG technique, one problem related to its signal is the susceptibility to noise derived from movements, known as motion artifacts (MA), which can degrade the quality of the heart rate estimation. This type of noise is very common during physical exercises—such as running, weightlifting, and cycling—and to mitigate the MA, a common approach is to process the raw PPG signal to maintain mainly the components related to the heart rate. The accelerometer extracts motion information during physical activity and aid in the preprocessing of the PPG signal when combined with digital filters.

However, when the PPG signal is affected by strong MA, which usually happens during intense workouts, e.g. cycling exercises, the approaches based on accelerometer and digital filters are insufficient to clean the signal adequately. In addition, for some types of workouts, the accelerometer may not represent the exercise intensity, like during cycling. Because of that, signal quality measures are usually included in the pipelines to avoid showing untrustworthy heart rate estimates when the PPG signal has poor quality or even to guide in correcting possible problems when the PPG signal is unreliable.

The patent document EP2992817A1, entitled “Electronic device and method for measuring vital signal”, published on Mar. 9, 2016, by SAMSUNG ELECTRONICS CO LTD, avoids capturing unreliable signals, such as the PPG, by measuring the vital signals only when the amount of motion is less than or equal to a defined threshold thus preventing computing the heart rate in such moments. However, the method at the patent will not compute vital signals when there is a higher level of motion intensity, which is exactly the goal of the present invention. The approach defined at EP2992817A1 limits the use of the method on smartwatches, considering that users expect to monitor their heart rate in scenarios of intense movements, for instance, running exercises. On the other hand, the proposed invention can agnostically leverage the PPG signal quality index (SQI) available in modern smartwatches. Thus, it can correct the heart rate only when the PPG signal is unreliable, regardless of the intensity of the motion.

The patent document US2017127960A1, entitled “Method and apparatus for estimating heart rate based on movement information”, published on May 11, 2017, by SAMSUNG ELECTRONICS CO LTD, provides two regression models used to estimate the heart rate. The first regression model measures the correlation between the average heart rates and average motion information of many users, and the second is based on the correlation between heart rates and previous motion information of the user to estimate the heart rate. If the pipeline determines that personalization will be applied, previous moving information is passed to the second regression model to be personalized. Different from US2017127960A1, the present invention defines only one model to estimate the heart rate using parameters optimized by machine learning strategies. Also, neither the barometer sensor nor its data were mentioned in US2017127960A1.

The patent document CN112790752A, entitled “Heart rate value correction method and device and electronic equipment”, published on May 14, 2021, by VIVO MOBILE COMMUNICATION, provides a method that estimates the motion state of a user by the information from inertial or barometric sensors and uses it to correct the heart rate. Using the motion information, it selects the motion state of the user, which can be categorized as high or low, and corrects the first heart rate if it was not in the valid frequency range considering the spectral information. Similarly to the present invention, document CN112790752A may compute the motion state with the aid of a barometric sensor. However, the motion state in document CN112790752A is preset, and not continuous such as our invention and any preprocessing or transformation is applied to the barometer signal. Moreover, CN112790752A suggests a heart rate correction using spectral information. In the proposed method, the heart rate is estimated using a model which learns the parameters based on the reliable heart rate to improve the result of the PPG-based algorithm and does not use any information related to the highest peak value of the signal spectrum. The use of spectral information in many cases may be misleading due to the poor PPG signal quality. Therefore, the present invention avoids the use of the PPG signal information when it can be highly affected by motion artifacts, instead trusting in barometer information to correct the gaps between the reliable heart rate estimated during a workout session.

Bearing this in mind, the main goal of the present invention is to adjust the heart rate estimation of workout sessions in which the information of the altitude is relevant for the exercise, such as outdoor cycling, when the PPG signal quality is unreliable by using an exponential model that runs at the end of a workout session. This model considers the barometer information to identify points of increase and decrease in heart rate and estimates the intensity of a workout. By using this information, the heart rate can be estimated to replace the unreliable heart rate prediction of a PPG-based algorithm.

The selection of this type of exercise comes from the fact that workouts that have variation in altitude and inclination, such as outdoor cycling or hiking, are very popular but still have a considerable error in the heart rate estimation compared to running/walking due to noise derived from the non-plane terrain, which may increase motion artifacts. Considering the difficulty in improving heart rate estimates for these workouts using just the accelerometer, usually, these exercises are put aside despite the strong interest of the users. We observed that for the workouts in which the altitude impacts the heart rate, the barometer signal can bring relevant information that can be used to correct the heart rate in the PPG unreliable regions, thus improving the heart rate estimation accuracy.

### SUMMARY

The present invention describes a method to predict the heart rate when the PPG signal from a wearable device is affected by motion artifacts during any activity in which there is a change in altitude during the workout. The method is composed of five blocks: preprocessing of the barometer signal, computation of the workout intensity, downsampling, prediction of the heart rate using an exponential approximation model, and the improvement of the model parameters by using an optimizer.

### BRIEF DESCRIPTION OF THE DRAWINGS

The objectives and advantages of the invention will become clearer through the following detailed description of the example and non-limitative drawings presented at the end of this document.

FIG. 1 presents the heart rate prediction of a reference device and a smartwatch during an outdoor cycling workout according to an exemplary embodiment of the present invention.

FIG. 2 presents the pipeline for heart rate prediction according to an exemplary embodiment of the present invention.

FIG. 3 depicts the influence of the terrain slope in the heart rate variation according to an exemplary embodiment of the present invention.

FIG. 4 illustrates the reference heart rate and barometer signal during an outdoor cycling workout according to an exemplary embodiment of the present invention.

FIG. 5 presents the convergence chart for the algorithm learning according to an exemplary embodiment of the present invention.

FIG. 6 shows an embodiment of the method to correct the heart rate of the unreliable parts of the PPG signal according to an exemplary embodiment of the present invention.

FIG. 7 is an illustration of an exemplary embodiment of the present invention.

### DETAILED DESCRIPTION

A lightweight method for computing the heart rate in scenarios where variation in altitude can cause increases or decreases in heart rate, such as outdoor cycling exercises or hiking in mountainous places is proposed. In such exercises, unreliable heart rate prediction can occur because of regions in PPG signal with a low signal quality and the proposed model can be applied to correct these regions, taking into consideration the reliable parts of the heart rate signal.

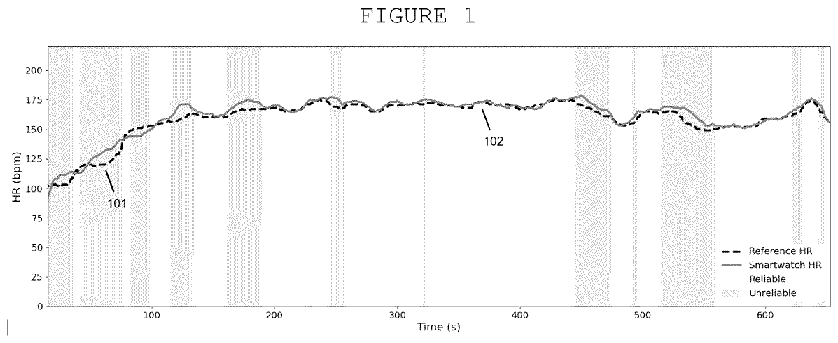

One example of a workout with issues in the PPG signal is depicted in FIG. 1. In some regions of the workout, such as the gray region 101, the difference between the reference heart rate and the heart rate from a smartwatch is high, which indicates that the quality of the PPG in that region is not reliable and could not be used to estimate the heart rate with precision. In other regions, as in the white region 102, the heart rate from the wearable device and the reference are close, which suggests a good signal quality.

Considering the PPG signal quality, usually embedded in the processing pipeline, the method is applied when the signal is unreliable to avoid the problem of untrustworthy heart rate predictions, by aiding the PPG-based algorithm when it does not have enough information for a reliable estimation. For that, the proposed method learns a model that relates the workout intensity of an exercise, computed using the barometer signal, with the reliable heart rate estimates from the PPG-based algorithm, combining them for a more accurate prediction.

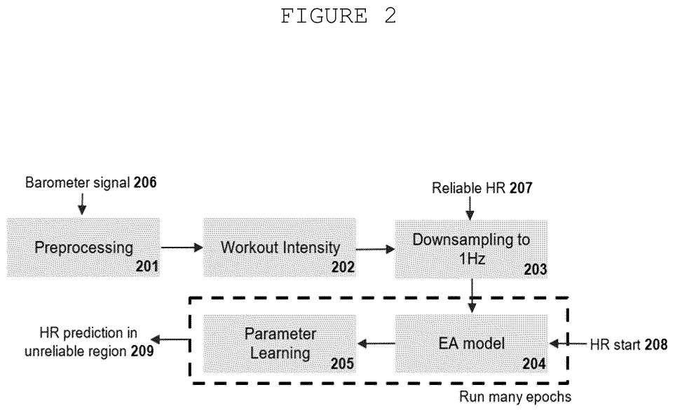

In a preferred embodiment, the method is composed of five blocks: (1) the barometer preprocessing block 201, (2) the computation of workout intensity 202, (3) downsampling block 203, (4) the Exponential Approximation (EA) model 204 for post-processing the unreliable heart rate and (5) the parameter learning by a machine learning technique 205. The pipeline is depicted in FIG. 2, together with the description of the inputs for each one of the blocks.

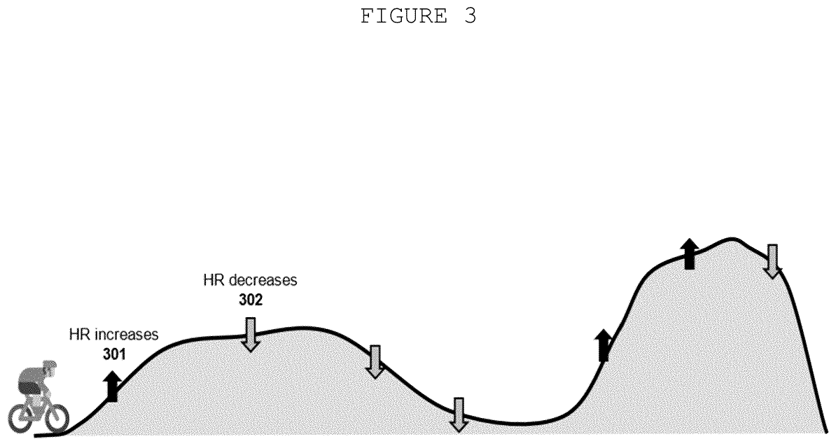

Block 201 receives a wearable device's raw barometer signal 206 and preprocesses the input signal. The barometer sensor measures the atmospheric pressure of a place. The idea behind the use of this signal is that the effort to go uphill is higher than staying at the same altitude or going downhill maintaining a constant speed. Take, for example, an outdoor cycling workout, as illustrated in FIG. 3, when a person starts cycling uphill, the heart rate will increase fast (e.g., 301), and after they reach a constant altitude or ride downhill, the heart rate will start decreasing (e.g., 302). Considering this, the atmospheric pressure can give information about the altitude during an exercise and, consequently, bring some insights about the variation in the heart rate in such scenarios.

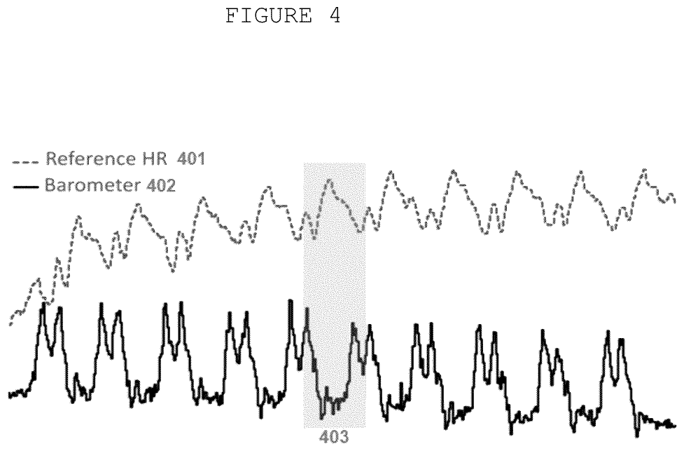

Observing the signals in FIG. 4, it is possible to see a clear relationship between the heart rate 401 and the barometer signal 402 when a subject is performing a cycling exercise. The workout in FIG. 4 starts on flat ground and in the regions in which the barometer is lower, i.e. high altitudes, the subject rides downhill, manifesting an increase in the barometer value. Oppositely, when the subject goes uphill in the workout, the barometer decreases and the heart rate becomes higher, showing an inversely proportional relationship, as highlighted in the region 403. The total number of laps in the presented workout is ten.

### Preprocessing Block

The preprocessing block 201 is responsible for making the necessary modifications to aid in the computation of the workout intensity (WI) 202 needed to estimate the heart rate. It may include digital filters to clean and improve the barometer signal, normalizations, or other types of processing steps. Moreover, considering the relationship between the heart rate and barometer, the preprocessing block receives the barometer signal and reverses it in the y-axis to have a positive correlation. It may also remove the mean value of the entire barometer signal to better capture the small pressure differences.

### Workout Intensity Computation

To compute the WI 202, the present invention uses a weighted moving average to transform the preprocessed barometer (baro) in the barometer intensity (BI) information according to the following equation (1), in which the initial BI is zero. In one exemplary embodiment, the forgetting factor λ was set to 0.998, but any other value between 0 and 1 can be applied. This approach can result in a smoother version of the barometer signal. Usually, λ is configured between 0.960 and 0.999 for dynamic systems.

$$BI ( t ) = λ * BI ( t - 1 ) + ( 1 - λ ) * baro ( t ) \qquad ( 1 )$$

The derivative of the BI, BI_{Δ}, is computed to extract the regions of the signal where there is a difference in altitude. It is important to consider that during a downhill cycling workout the heart rate usually starts to decrease. To take it into account, the WI module transforms values below zero to zero after the computation of the derivative of the barometer intensity (equation 2), considering that values below zero represent regions of flag terrain or downhills, defining the WI for each sample. Afterwards, this value may be multiplied by a factor A to amplify the difference between points thus improving the next steps of the method. This value can be fixed for all subjects or based on a demographic parameter. For the present invention, the value of A is 40000 empirically.

$$WI ( t ) = A * max ( 0 , {BI}_{Δ} ( t ) ) . \qquad ( 2 )$$

It is important to mention that other methods can be used to smooth the barometer values instead of the weighted moving average and will not have a significant impact on the final output.

### Downsampling to 1 Hz and Reliable HR

Downsampling module (203) is responsible for downsampling the WI and the reliable heart rate 207 from the sampling rate of the smartwatch, e.g., 25 Hz, to 1 Hz to allow a smaller buffer of data. The downsampling module is optional, i.e. the signals may be processed in the original sampling rate, and the proposed method also may be implemented using other sampling rates.

Reliable heart rate is obtained by using the heart rate computed from the PPG signal and its corresponding quality index to isolate the reliable parts of the signal. The PPG signal used as input of this method is expected to be preprocessed, using methods such as bandpass filters, Normalized Least Mean Squares, or any other kind of filter that may decrease motion artifacts and improve the quality of the signal. Moreover, any SQI algorithm can be used to estimate the reliability of the PPG for the present pipeline. In the present invention, the SQI was computed based on the difference between the heart rate signal from the PPG-based algorithm and the reference heart rate instead of using the PPG SQI from the chosen device.

### Exponential Approximation Model

The EA model block 204 receives as input: the reliable heart rate, the WI, and the first heart rate of the workout (HR start) 208 to learn the parameters of an exponential model which relates the heart rate in a time step t with the intensity of the movement in that instant. The use of an EA model to correct the heart rate comes to the fact that the cardiovascular kinetics during a workout may resemble an exponential function over time. This model is defined following the subsequent equation, in which the HR(t+1) depends on the HR_{stable}, that is an estimate of where the heart rate at that moment would stabilize and an exponential component raised to the power of −1/τ. The parameter τ is responsible for determining how fast the heart rate will rise or fall. This is a recurrent model, i.e., we must know HR(t) to estimate HR(t+1) and so on:

$$HR ( t + 1 ) = {HR}_{stable} - [ {HR}_{stable} - HR ( t ) ] * {e}^{- \frac{1}{τ}} . \qquad ( 3 )$$

HR_{stable} in a given moment is defined in Equation 4 and depends on the value of the WI, μ, HRₘₐₓ and HR_{Δ}. The multiplication by a is only for parameter learning purposes. In an exemplary embodiment, it is defined as α=0.001.

$${HR}_{stable} = {HR}_{Δ} + μ * α * WI ( t ) * ( {HR}_{max} - {HR}_{Δ} ) . \qquad ( 4 )$$

The model proposed here depends on 3 trainable parameters to predict the HR: μ,τ, and HR_{Δ}. μ is a parameter that controls the weight of the WI for the workout. HR_{Δ} plays a similar role to the heart rate during the resting state and estimates the rest heart rate of a workout. τ is related to how fast the exponential function will converge to the stable value, as explained previously. HRₘₐₓ can be computed using any approach available in the state of the art or can be fixed for all the subjects. In an exemplary embodiment, HRₘₐₓ=220−age.

### Parameter Learning

To train the parameter of the model, the quality of the PPG signal during the workout is considered. If the heart rate obtained from the PPG-based approach is reliable, the method will use the respective samples to train the parameters of the model for each workout session. So, the first step of this module is to receive the reliable heart rate and use it to tune the initial parameters of the μ, τ, and HR_{Δ}.

The mean absolute error (MAE) was chosen to compute the error between the heart rate predicted by the model, ŷ, and the heart rate from the reliable region, y, during the model training following the MAE definition below.

$$MAE ( y , \hat{y} ) = \frac{1}{N} {{\sum }^{N - 1}}_{i = 0} \vert {y}_{i} - \hat{{y}_{i}} \vert \qquad ( 5 )$$

For adapting the parameters θ={μ,τ,HR_{Δ}}, some runs of a gradient descent algorithm are performed using the derivatives of the MAE (equation 6) throughout the epochs considering the reliable part of the signal of the workout as a whole. An epoch is defined as one run of the algorithm in the whole workout signal.

$$\begin{matrix}\frac{\partial MAE ( y , \hat{y} )}{\partial Θ} = \frac{\partial }{\partial Θ} [ \frac{1}{N} {{{\sum }^{N - 1}}_{i = 0}}_{}^{} ( {y}_{i} - \hat{{y}_{i}} ) ] \\ = \frac{1}{N} {{{\sum }^{N - 1}}_{i = 0}}_{}^{} \frac{\partial ( {y}_{i} - \hat{{y}_{i}} )}{\partial Θ} \\ = \frac{1}{N} {{{\sum }^{N - 1}}_{i = 0}}_{}^{} \frac{\partial {y}_{i}}{\partial Θ} - \frac{\partial \hat{{y}_{i}}}{\partial Θ} \\ = \frac{1}{N} {{\sum }^{N - 1}}_{i = 0} {-}_{}^{} \frac{\partial \hat{{y}_{i}}}{\partial Θ} .\end{matrix} \qquad ( 6 )$$

One problem regarding using the MAE is that it is not differentiable in zero, which is an obstacle to the use of the gradient descent strategy. Considering this, the derivatives for yᵢ−ŷᵢ>0 are computed. When yᵢ−ŷᵢ<0, the signal of the derivatives is inverted.

From equations 3 and 4, the derivative for the minimization of the MAE fox the parameters μ, τ, and HR_{Δ} to be applied in the gradient descent strategy were:

$$\frac{\partial HR ( t + 1 )}{\partial μ} = α * WI ( t ) [ {HR}_{Δ} - {HR}_{max} ] {e}^{- \frac{1}{τ}} ; \qquad ( 7 )$$

$$\frac{\partial HR ( t + 1 )}{\partial τ} = [ HR ( t ) - {HR}_{stable} ] [ \frac{{e}^{- \frac{1}{τ}}}{{τ}^{2}} ] ; \qquad ( 8 )$$

$$\frac{\partial HR ( t + 1 )}{\partial {HR}_{Δ}} = [ 1 - α * μ * WI ( t ) ] [ 1 - {e}^{- \frac{1}{τ}} ] . \qquad ( 9 )$$

The initial parameters of the proposed EA model were μ=60, τ=60, and HR_{Δ}=80, and the learning rate for the gradient descent algorithm was defined as 3, using an early stopping patience of 5 epochs.

Moreover, to improve the learning process, an online learning rate adaptation is used to dynamically update the learning rate during the gradient descent strategy. The hypergradient learning rate used in the proposed pipeline was 0.1.

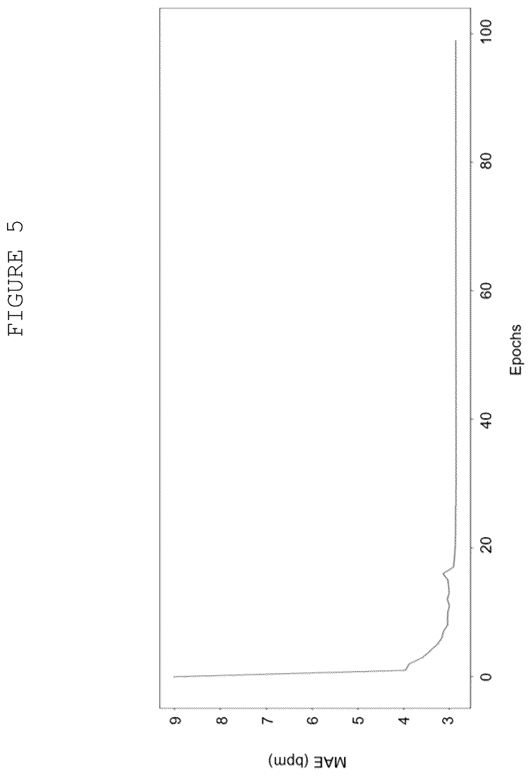

Block 204 and 205 can be run multiple times until the parameters of the model converge to the minimum error, as shown in FIG. 5.

After all epochs, the model with the lowest MAE is used to predict the heart rate in the unreliable region and the parameters are stored to be used as initial values for the next workouts of the same subject. This approach improves the performance of the model as the parameters of the previous workout sessions tend to be close to the ones that will be adapted in the current workout session, considering that some of them reflect the cardiovascular status of the user (e.g., resting heart rate and speed of the increase in heart rate). Moreover, in an alternative embodiment, parameter storing can be removed from the pipeline, thus using the same initialization mentioned previously. However, the results may be less consistent without parameter storing. Also, the proposed method includes a smoothing function in the transition between the predicted and reliable heart rate that starts some seconds before the beginning of the next reliable region.

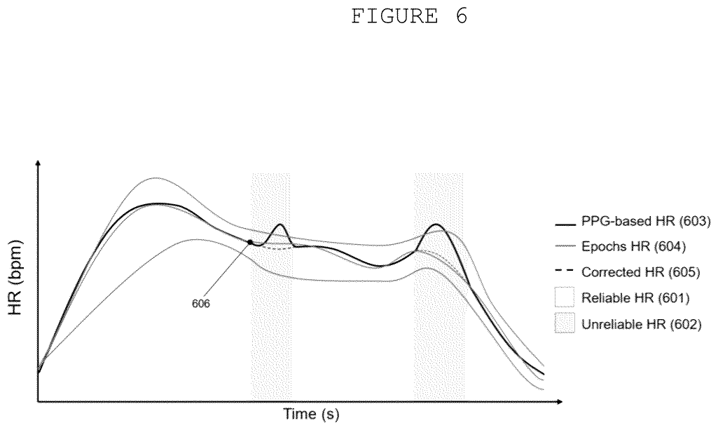

FIG. 6 depicts an exemplary embodiment where the steps of the invention can be described in summary as:
Identification of reliable 601 and unreliable 602 parts of the HR computed by PPG 603 based on an SOI algorithm;
Obtaining HR start and WI for HR estimation using the EA model;
For each epoch, estimating the HR 604, and updating the EA parameters by minimizing MAE in the reliable part;
After estimating heart rate for all epochs, using the trained parameters to estimate heart rate in the unreliable region (605) with the last reliable sample of the previous block (606) as the initial point.
   Estimating Workout Intensity from Barometer Signal to Post-Process Heart Rate Predictions for Outdoor Running

Accelerometer signals can be used to estimate workout intensity for outdoor running by measuring the amount of movement a person is doing during the exercise, and this workout intensity can be used to estimate the heart rate when PPG signals are affected by movement noise preventing reliable HR estimates. Thus, the HR predictions can be post-processed to reduce errors due to corrupted or poor-quality PPG signals.

However, in situations where there is strong terrain elevation variability, the amount of movement measured from the accelerometer signals might not be enough to estimate the workout intensity. This might happen because the person might run slower when the inclination is positive and faster when it is negative which can impact the HR predictions based on workout intensity from the accelerometer signals. The barometer signal is related to terrain elevation, which can be used to improve the workout intensity estimation. Hence, the real workout intensity can be defined as a combination of the workout intensity estimated from the accelerometer signal and the workout intensity estimated from the barometer signal as defined in Equation (2). This way, the model can account for terrain elevation changes and produce more accurate HR estimates.

Estimating Workout Intensity from Barometer Signal for Real-Time Predicting Heart Rate for Outdoor Cycling

For estimating the heart rate in real-time using the barometer signal, the workout intensity can be defined in a similar way to Equations (1) and (2). However, there are some changes needed to be done as the mean of the barometer signal is not known a priori. As we cannot subtract the mean value, we need to wait a warm-up time for the workout intensity estimated from the barometer signal to be ready to be used. After that, if the barometer signal decreases, then the workout intensity increases and vice versa. The remaining post-processing model can be used in an online learning framework for predicting the heart rate in real-time.

To evaluate the effect of the method, experiments were carried out by using an outdoor cycling dataset containing 93 subjects with a total of 197 valid workouts for the entire dataset. This dataset contains data from 82 men and 11 women with a mean±standard deviation age of 34±8.

For the data collection, all, subjects wore a Polar H10 chest strap to measure the reference heart rate and a Galaxy Watch 5 to collect the heart rate using its PPG-based algorithm and also get the barometer signal for the WI computation. Each outdoor cycling workout was composed of 10 laps in an asphalt terrain circuit with inclination variation, with a total distance of 4,370 meters. The subjects performed between 2 and 4 workouts each.

The SQI of the signals was defined considering the heart rate from the PPG-based algorithm. If the difference between the heart rate from the wearable device and the reference at a point in time was higher than 3 bpm, the quality of the PPG signal was considered unreliable, and the proposed method was used to correct the heart rate estimates based on this part of the signal. Other thresholds may be used to define the signal quality. A difference under 5 bpm for reliable heart rate is suggested to guarantee samples of good quality for the training step.

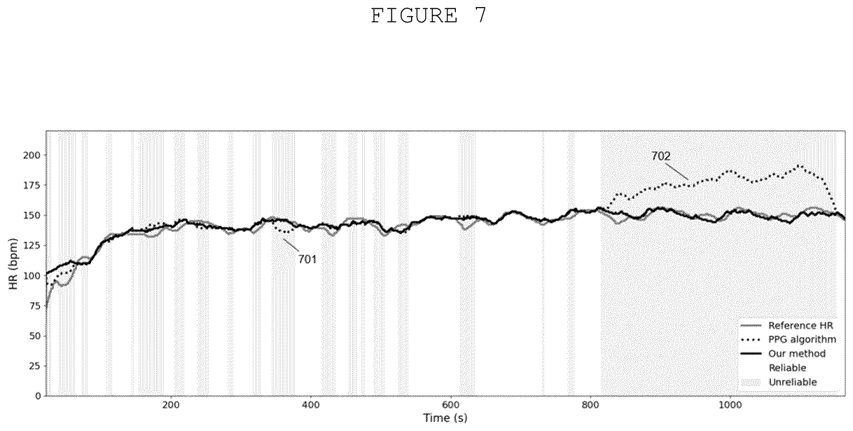

One example of an improvement in a workout can be seen in FIG. 7. It is possible to see that in some unreliable regions, e.g., 701 and 702, the heart rate from the PPG-based algorithm was far from the reference, but the method was able to correct the heart rate in such areas. The proposed pipeline decreased the MAE from 9.95 bpm to 3.07 bpm in this specific case, representing an improvement of 69%.

To evaluate the method, two metrics were selected: the MAE between the heart rate estimate obtained and the reference, and the pass rate (PR) which is defined as the percentage of the data points in which the absolute error between the method and reference is below 10 bpm. The percentage of improvement taking the MAE of the PPG-based algorithm as a baseline was also computed.

To compare the results of the EA model with lightweight classical methods, three approaches were implemented: Lasso regression, Random Forest, and Support Vector Machine (SVM). A sliding window of 20 seconds was used to predict the next heart rate. The previous reliable heart rate and WI were inputted in the models, SVM used a radial basis function kernel and the maximum depth of the Random Forest was set to 8. The results are presented in Table 1.

Table 1—Results of the PPG-based method compared to the proposed method and classical methods of the state of the art for the dataset with 197 cycling workouts. Values indicate the mean±standard deviation. The percentage of improvement considers the MAE value.

| | | | % |
|---|---|---|---|
| | MAE (bpm) | PR (%) | Improvement |
| PPG | 7.07 ± 8.21 | 79.01 ± 29.54 | — |
| algorithm | | | |
| EA model | 4.61 ± 3.83 | 87.04 ± 16.70 | 34.84 |
| Lasso | 6.39 ± 4.45 | 79.68 ± 19.30 | 9.68 |
| regression | | | |
| Random | 6.23 ± 6.78 | 81.40 ± 18.31 | 11.86 |
| Forest | | | |
| SVM | 6.30 ± 4.39 | 79.40 ± 19.33 | 10.96 |

It is possible to observe that the disclosed method is better than the methods used for comparison for at least 22 percentage points. The proposed method improved the heart rate estimation in approximately 35%, i.e., 2.46 bpm.

Although the present invention has been described in connection with certain preferred embodiments, it should be understood that it is not intended to limit the disclosure to those particular embodiments. Rather, it is intended to cover all alternatives, modifications and equivalents possible within the spirit and scope of the disclosure as defined by the appended claims.

What is claimed is:

1. A method for estimating and correcting heart rate in exercises using barometer signal in a wearable device, the method comprising:
receiving a barometer signal (baro(t));
computing barometer intensity (B(t)) as:
BI(t)=λ*BI(t−1)+(1−λ)*baro(t), where λ is a forgetting factor;
computing workout intensity (WI(t)) as:
WI(t)=λ·max(0,BI_{Δ}(t)),
where BI_{Δ} is a derivative of BI(t) that identifies regions of the barometer signal where there is a difference in altitude, A is a factor to increase the amplitude of the workout intensity;
receiving a reliable heart rate (HR) region and an unreliable HR region and the first HR of a workout session;
for the reliable HR region, the method further comprises:
computing the mean absolute error (MAE) between the HR predicted by the model (ŷ) and the reliable HR (y) as follows:
$$MAE ( y , \hat{y} ) = \frac{1}{N} {\sum }_{i = 0}^{N - 1} \vert {y}_{i} - \hat{{y}_{i}} \vert ;$$
using gradient descent for adapting a set of parameters θ={μ,τ,HR_{Δ}}, such that:
$$\frac{\partial MAE ( y , \hat{y} )}{\partial Θ} = \frac{1}{N} {\sum }_{i = 0}^{N - 1} - \frac{\partial \hat{{y}_{i}}}{\partial Θ} ,$$
where N is the number of epochs considered;
storing the set of parameters θ={μ,τ,HR_{Δ}} with the lowest MAE; and
for identifying the heart rate (HR(t+1)) in the unreliable HR region, the method further comprises:
feeding HR_{stable}, WI(t) and the first HR to a recurrent model, wherein:
$$HR ( t + 1 ) = {HR}_{stable} - [ {HR}_{stable} - HR ( t ) ] * {e}^{- \frac{1}{τ}} ;$$
$${HR}_{stable} = {HR}_{Δ} + μ * α * WI ( t ) * ( {HR}_{max} - {HR}_{Δ} ) ,$$
where τ determines how fast HR will rise or fall, μ is a parameter that controls the weight of WI(t), α is the learning parameter, HRΔ is the estimated rest HR of the workout session and HRₘₐₓ is a maximum pre-set theoretical heart rate value.

2. The method as in claim 1, wherein the reliable HR region is obtained by isolating reliable parts of a PPG signal of the wearable device.

3. The method as in claim 1, wherein the reliable HR region is defined by a signal quality index (SQI).

4. The method as in claim 1, wherein the barometer intensity (BI) and the reliable HR are downsampled.

5. A wearable electronic device comprising:
a processor;
a PPG sensor to measure a PPG signal;
a barometer; and
a memory to store computer readable instructions that, when executed by the processor, causes the processor to perform the method as defined in claim 1.
